(19) United States
(12) Patent Application Publication
(10) Pub. No.: US 2009/0202651 A1
(43) Pub. Date: Aug. 13, 2009

(54) Antigen specific fluorescent nanoparticles

(76) Inventors: Michael A. Moody, Durham, NC (US); Barton F. Haynes, Durham, NC (US)

Correspondence Address: NIXON & VANDERHYE, PC, 901 NORTH GLEBE ROAD, 11TH FLOOR, ARLINGTON, VA, 22203, US

(21) Appl. No.: 12/310,480

(22) PCT Filed: Aug. 24, 2007
(86) PCT No.: PCT/US07/18683

§ 371 Date: Apr. 9, 2009

Related U.S. Application Data
(60) Provisional application No. 60/840,423, filed on Aug. 28, 2006.

(51) Int. Cl. A61K 39/21; A61K 9/16; C12N 5/08; A61P 31/18

(52) U.S. Cl. 424/491; 435/325; 424/208.1; 424/493; 424/490

(57) ABSTRACT

The present invention relates, in general, to nanoparticles and, in particular, to nanoparticles coated with, for example, peptides, proteins, and/or carbohydrates, and to methods of producing and using same. The invention further relates to kits comprising the coated nanoparticles.

This application claims priority from U.S. Provisional Application No. 60/840,423, filed Aug. 28, 2006, the entire contents of that application being incorporated herein by reference.

This invention was made with Government support under Grant No. AI 0678501 awarded by the National Institutes of Health. The Government has certain rights in the invention.

### TECHNICAL FIELD

The present invention relates, in general, to nanoparticles and, in particular, to nanoparticles coated with, for example, peptides, proteins, and/or carbohydrates, and to methods of producing and using same. The invention further relates to kits comprising the coated nanoparticles.

### BACKGROUND

More than twenty years after the recognition of the Acquired Immunodeficiency Syndrome (AIDS) pandemic, human immunodeficiency virus type 1 (HIV-1) continues to spread unchecked through the world. At present the World Health Organization estimates 1% of all adults aged 15-49 years are infected with HIV-1. Numerous vaccines against HIV-1 are in clinical trials, but none has yet been shown to elicit effective and long-standing protective anti-HIV-1 immunity. A critical question for vaccine development is why long-lasting and broadly neutralizing responses against native HIV-1 are not induced by HIV-1 immunogens that generate robust immune responses to the immunogens themselves. The availability of reagents that allow for the labeling, detection, and isolation of components of the immune system having reactivity to specific portions of HIV-1 would facilitate the understanding of this roadblock to vaccine development.

More generally, determining the specificity of molecular recognition sites is an important problem for immunology. Although several techniques are currently used, more efficient and more flexible methods are desirable. The detection of receptor specificity using peptides or proteins in plate-based assays has the advantage of relatively inexpensive reagents and the capacity for high throughput but lacks the ability to look at the level of the individual cell. Antibody capture assays using cell suspensions are capable of enumerating individual cells but do not allow for the sorting and selection of those cells for further study. Staining of the cells using antigen-specific reagents created from labeled streptavidin allows for flow cytometric measurements and sorting but requires the use of biotinylated antigens in their production. Thus, it is desirable to have a detection reagent that is useful across assays and that is produced from less specialized and relatively inexpensive materials.

Semiconductor crystal nanoparticles (commonly referred to as quantum dots) are a small crystals of cadmium selenide, indium arsenide, or other such materials that, when excited by high energy photons, emit lower energy photons (i.e., fluoresce) at defined wavelengths (Rosetti and Brus, J. Phys. Chem. 86(23):4470-4472 (1982)). The size of a semiconductor crystal nanoparticle determines the wavelength of emitted fluorescence, thus such nanoparticles can be selected for desirable optical properties based on their size. They are relatively photostable compared with organic or protein-based fluorochromes. Furthermore, they can be coated with a variety of materials and can, therefore, be engineered with surface properties appropriate for the desired application. These surface coatings include, but are not limited to, non-polar lipid coatings and amphipathic molecules that allow for greater water solvation of the particles. The surfaces can also be derivatized with reactive groups that facilitate the conjugation of the nanoparticles to other materials. (Smith et al, Ann. Biomed. Eng. 34(1):3-14 (2006).)

### SUMMARY OF THE INVENTION

The present invention relates generally to nanoparticles. More specifically, the invention relates to nanoparticles attached to which are, for example, peptides, proteins, and/or carbohydrates. The invention also relates to methods of producing and using such nanoparticles and to kits comprising same.

Objects and advantages of the present invention will be clear from the description that follows.

### BRIEF DESCRIPTION OF THE DRAWINGS

FIG. 1. Model of sphere with antigens projecting from surface.

FIG. 2. Diameter of sphere.

FIG. 3. Model of hexagon tiling of plane with central spots representing antigen attachments.

FIG. 4. Sensogram of binding of 2F5-epitope nanoparticles to antibodies (control antibody: light blue, antibody with binding near 2F5 site: red, V3 antibody: dark blue, 2F5 antibody: green).

FIG. 5. Sensogram of binding of V3 loop nanoparticles to antibodies (control antibody: light blue, antibody with binding near 2F5 site: red, V3 antibody: dark blue, 2F5 antibody: green).

FIG. 6. Histogram of beads stained with nanoparticles detected on flow cytometry (stained control beads: grey, stained specific heads: solid line).

FIGS. 7-1 to 7-37. Novel fluorescent nanoparticle epitope-specific reagents for the detection of antigen specific B cells.

### DETAILED DESCRIPTION OF THE INVENTION

The present invention relates to nanoparticles attached to the surfaces of which are, for example, peptides, proteins, carbohydrates, or other molecules of interest. In a preferred embodiment, the nanoparticles bear on their surface specific antigens, for example, viral (e.g., HIV) or bacterial antigens.

Nanoparticles suitable for use in the invention include semiconductor crystal (e.g., quantum dot) nanoparticles comprising small crystals of, for example, cadmium selenide or indium arsenide. Nanoparticles comprising other core materials can also be used. For example, such cores can comprise a biodegradable polymer, persistent non-fluorescent material or ferromagnetic metal, or other material. (Astete and Sabliov, J. Biomater. Sci. Polym. Ed. 17(3):247-89 (2006), Thorek et al, Ann. Biomed. Eng. 34(1):23-38 (2006).)

The nanoparticles can be coated using a variety of methods including, but not limited to, covalent bond formation via the reaction of surface amino- or carboxyl-groups to peptides or proteins using standard peptide coupling reagents (e.g., 1-ethyl-3-(3-dimethylaminopropyl) carbodiimide hydrochloride). (Nakajima and Ikada, Bioconjug. Chem. 6(1):123-30 (1995).)

There are several ways for controlling the attachment of materials to the surfaces of nanoparticles. For small engineered molecules, the attachment site can be controlled by the synthetic pathway used to produce the small molecule. For example, a peptide can be manufactured such that protecting groups on reactive side chains can be left intact at the time of removal of the peptide from the resin support. The protected peptide, or derivatized nanoparticle, can then be reacted with an activation reagent and the two coupled in a highly regiospecific manner. The peptide can then be deprotected after attachment to the bead revealing the desired peptide epitope on the surface.

Alternatively, structures can be added to the molecule of interest to direct the coupling reaction along the desired pathway. For example, a peptide or protein can be constructed with additional residues at a desired location that can act as an attachment point. Thus carboxyl-derivatized nanoparticles can be activated and reacted with a peptide or protein that contains one or more lysine residues at a critical location that can act as the attachment point. This approach allows for regioselectivity and avoids the need for further deprotection steps.

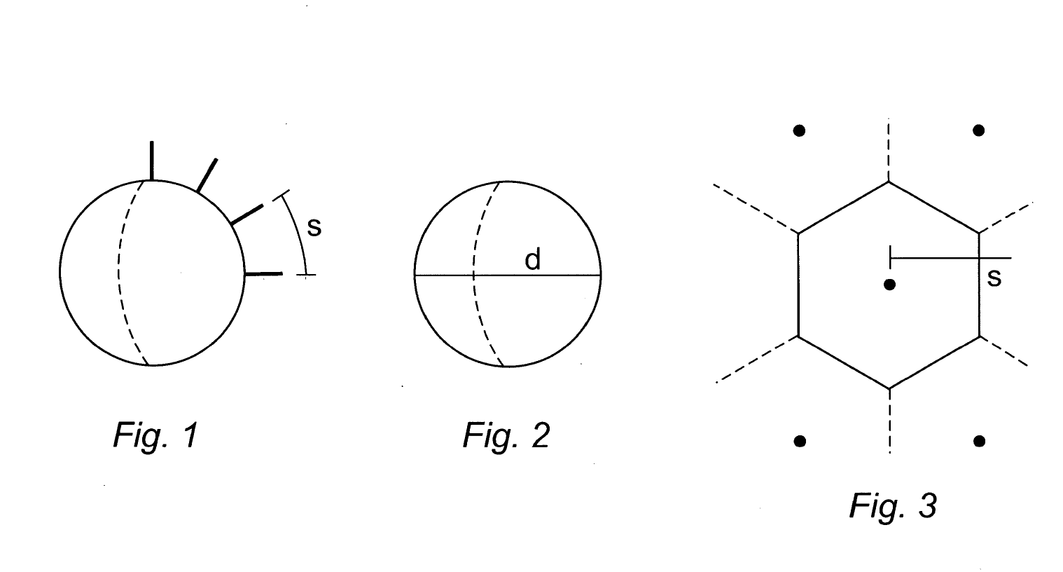

An important parameter for coating of the nanoparticles is the surface density of the molecule of interest. The desired spacing of the molecules on the surface can be different depending on the molecule and the application. Generally, a nanoparticle can be approximated as a sphere and the molecules of interest can be modeled as hairs projecting from the surface. Thus a determination of the desired spacing of molecules on the surface can be made and assigned a value s (FIG. 1).

A sphere has a surface area that is related to its diameter. Control the optical properties of the nanoparticle the size, and thereby the diameter, is effected during manufacture. Thus, for any given type of nanoparticle, the diameter is fixed. The surface area A for a sphere of diameter d (FIG. 2) is given by the formula in Eq. 1:

A=πd² Eq. 1

A spherical surface can be approximated by a Euclidian plane. Equidistant spacing of the molecules of interest on a plane can be achieved by tiling the plane with regular hexagons with the center of each hexagon representing the molecule of interest (FIG. 3). If the spacing of the molecules is given by s, then the apothem of the hexagon is given by ½ s. The area of the hexagon H can be calculated from the apothem using the following equation (where n=6 for a hexagon):

$$\begin{matrix}H = {n ( \frac{1}{2} s )}^{2} tan ( \frac{180}{n} ) \\ = \frac{3}{2} {s}^{2} tan 30 \\ = \frac{\sqrt{3}}{2} {s}^{2}\end{matrix} \qquad Eq . 2$$

The number of hexagons r needed to tile a plane of a given surface area can be calculated by Eq. 3:

$$r = \frac{A}{H} \qquad Eq . 3$$

The number of molecules of interest per nanoparticle, and thus the molar ratio of reactants during the preparation of the reagents, is given by r. Thus the spacing of molecules of interest on the nanoparticle can be controlled by the molar ratio of particles to molecules at the time of preparation.

Using these equations and this strategy, antigen-specific nanoparticles can be made with antigen surface densities that mimic those of a cell surface, of a virus particle, of the spacing of streptavidin as in the case of antigen-specific tetramers, or of any other arbitrary density desired.

The invention includes the above-described coated nanoparticles as well as the described coating method. In addition, the invention includes kits comprising the nanoparticles coated as described or uncoated and packaged with the peptide, protein, or other material to be attached to the particle surface. Such kits can include the components disposed within container means. Kits can also include ancillary reagents, including, for example, buffers.

Nanoparticles prepared in accordance with the invention have a wide array of potential applications including but not limited to the creation of novel immunogens with highly tailored surface properties. The nanoparticles can be used in the study of the immune system to determine correlates of immunity to HIV-1 or other infectious agents. For example, the method described allows for the delineation of the number and type of immune B cells circulating in the blood or present in accessible cellular compartments (lymph node, tonsil, spleen, bone marrow) that are present and that can respond to HIV-1 or other infectious agent and protect upon challenge. Furthermore, by using molecules of the protective epitopes of, for example, tetanus toxin, diphtheria toxin, anthrax toxin (protective antigen), hepatitis B envelope protein, influenza neuraminidase and hemagglutinin, etc., protective B cells in the blood or other bodily compartments or secretions can be analyzed to determine correlates of protective immunity. Using this strategy, it can be determined whether a person has adequate immunity or if they are at risk and at need for further immunization. The nanoparticles prepared as described herein can be used to create non-infectious immunogens capable of cross-linking cell surface antigens to more efficiently stimulate the immune system.

The present invention is exemplified by reference to commercially available nanoparticles having diameters from 15-25 nm. Based on previous work, a desired antigen spacing of 3 nm was selected. Using these parameters and the formulae above, the following values for r were calculated (Table 1):

TABLE 1
Calculations of desired molar ratios

| diameter of | | | | |
|---|---|---|---|---|
| sphere | area of sphere | spacing | area of hexagon | ratio r |
| 15 nm | 7.07 × 10¹⁶ m² | 3 nm | 7.79 × 10¹⁸ m² | 90.9 |
| 20 nm | 1.26 × 10¹⁵ m² | 3 nm | 7.79 × 10¹⁸ m² | 161 |
| 25 nm | 1.96 × 10¹⁵ m² | 3 nm | 7.79 × 10¹⁸ m² | 252 |

Certain further aspects of the invention are described in greater detail in the non-limiting Example that follows.

### EXAMPLE

### Experimental Details

Materials: nanoparticles derivatized with reactive carboxyl groups on the surface (available from multiple suppliers (e.g., Invitrogen and Evident Technology))
molecule of interest (peptide, protein, carbohydrate, etc.) with reactive amino group available
1-ethyl-3-(3-dimethylaminopropyl) carbodiimide hydrochloride
10 mM borate buffer (pH 8.3)
phosphate buffered saline with 0.02% w/v sodium azide
appropriate size stir bar
glass test tube
30,000 molecular weight cutoff concentrator (e.g., Millipore Centriprep)

Procedure:
1. Prepare stock of 1-ethyl-3-(3-dimethylaminopropyl) carbodiimide hydrochloride at 10 mg/mL (52 mM) in 10 mM borate buffer (pH 8.3).
2. Place nanoparticles at 8 μM into the glass tube with the stir bar.
3. Add 52 mM 1-ethyl-3-(3-dimethylaminopropyl) carbodiimide hydrochloride stock to the tube in the desired ratio (r). For example, if r is 150, add 150 fold excess.
4. Incubate at room temperature in the dark for one hour with gentle stirring.
5. Dissolve the molecule of interest at 2 mM in 10 mM borate buffer (pH 8.3).
6. Add 2mM molecule of interest to the tube in the desired ratio (r).
7. Incubate at room temperature in the dark for two hours with gentle stirring.
8. Work up the sample by placing the reaction mixture in a concentrator and exchanging the material five times into the final buffer (PBS with azide).
9. Determine final volume and use this to calculate a final concentration of particles based on 100% recovery.

In cases where the molecule of interest is insoluble in borate buffer, other aqueous, non-amine buffers (such as PBS) can be used. If required, organic solvents (such as DMSO) can be used. This approach does not require the use of carboxyl-derivatized nanoparticle and amines for reactivity. It can be used in the opposite orientation provided that there are no other reactive amino groups on the molecule of interest or a protection-deprotection strategy is employed. This strategy is not limited to the formation of amide bonds but can utilize other surface attachment strategies as they become available.

### Results

Nanoparticles were prepared using the method described above; one labeled with a peptide derived from HIV-1 that is specific for the recognition site of the 2F5 monoclonal antibody QQEKNEQELLELDKWASLWN, and another labeled with an HIV-1 envelope V3 loop peptide that is recognized by the F39F antibody TRPNNNTRKSIHIGPGRAFYATE. These were tested in two model systems.

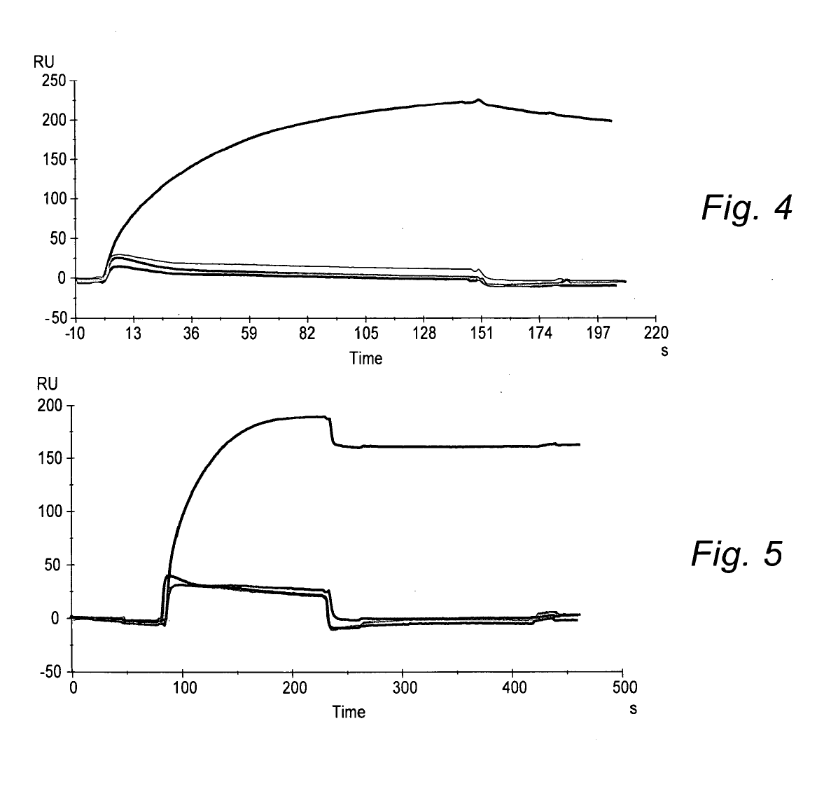

Surface plasmon resonance (SPR) studies were used to determine the specificity of binding of the nanoparticles to antibodies bound to the detection surface. FIG. 4 is a sensogram from the SPR study of the 2F5-epitope labeled peptide run against four antibodies (P3X63 (control antibody), F39F (V3 antibody), 5A9 (antibody with binding near 2F5 site) and 2F5)—it clearly shows specific binding of the nanoparticles to 2F5 and absence of binding to three other antibodies. Similarly, the V3 loop-labeled nanoparticles were tested on the same four antibodies. The results clearly show that the nanoparticles specifically bind to the anti-V3 antibody and do not bind to the others (FIG. 5).

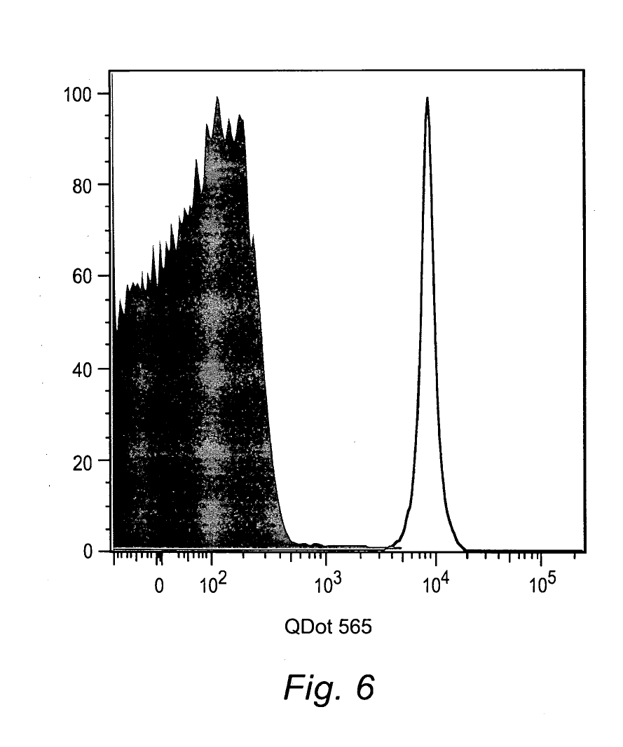

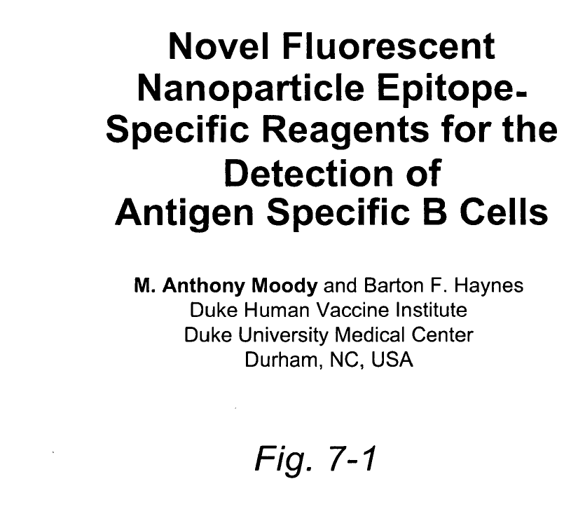

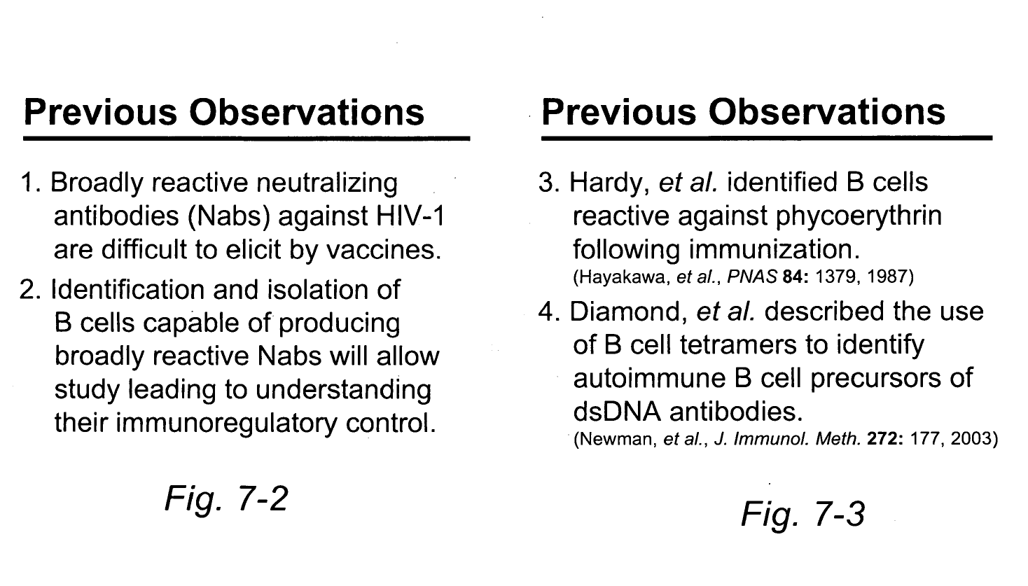

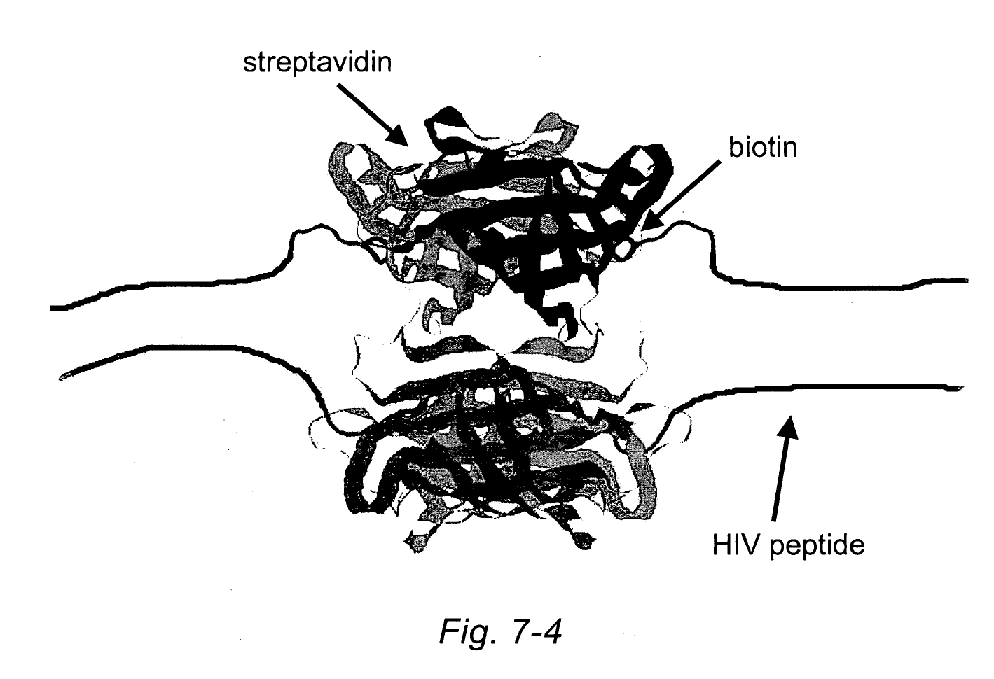

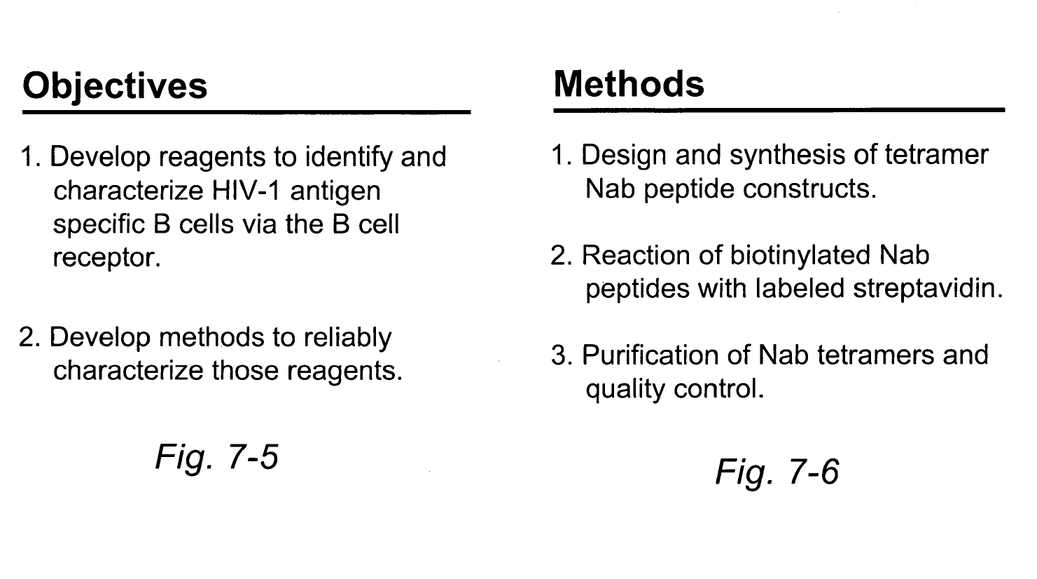

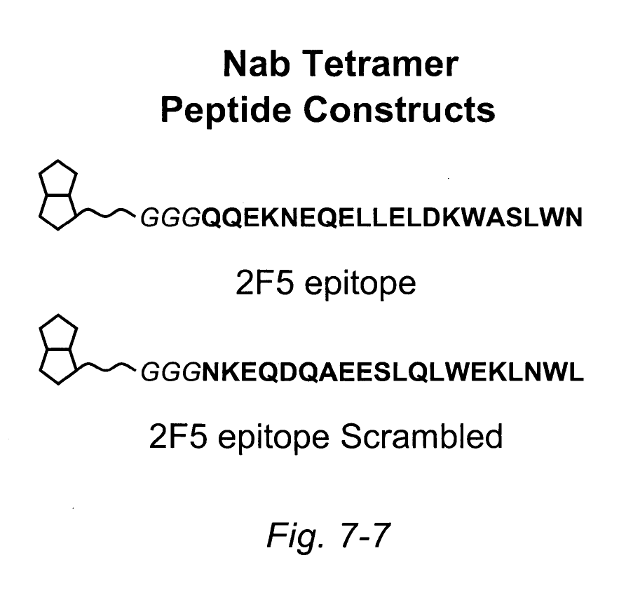

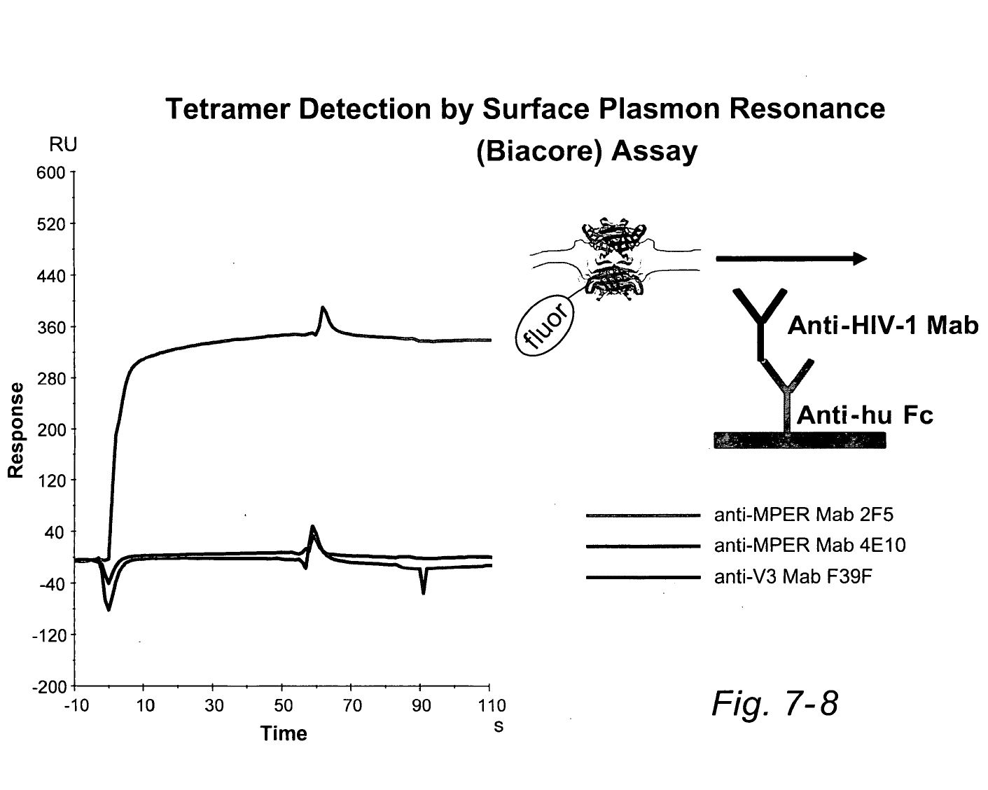

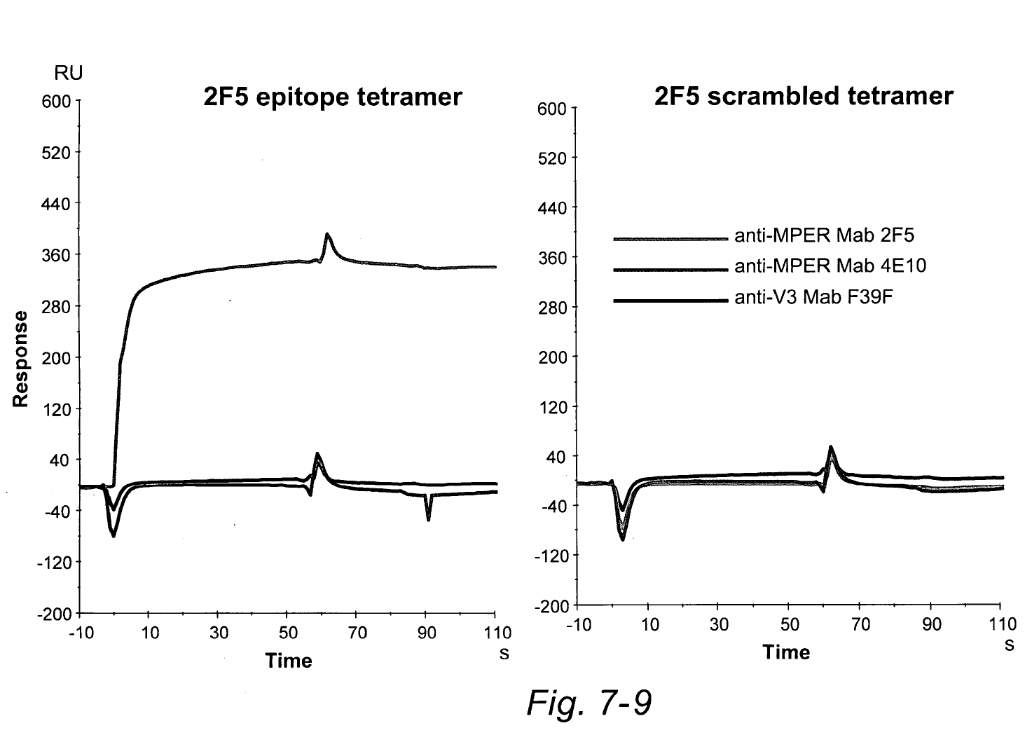

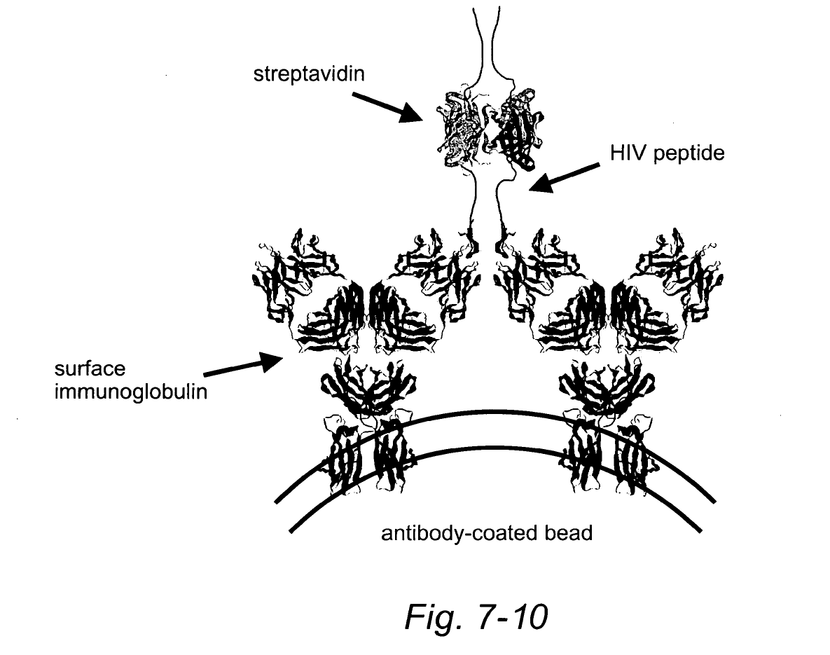

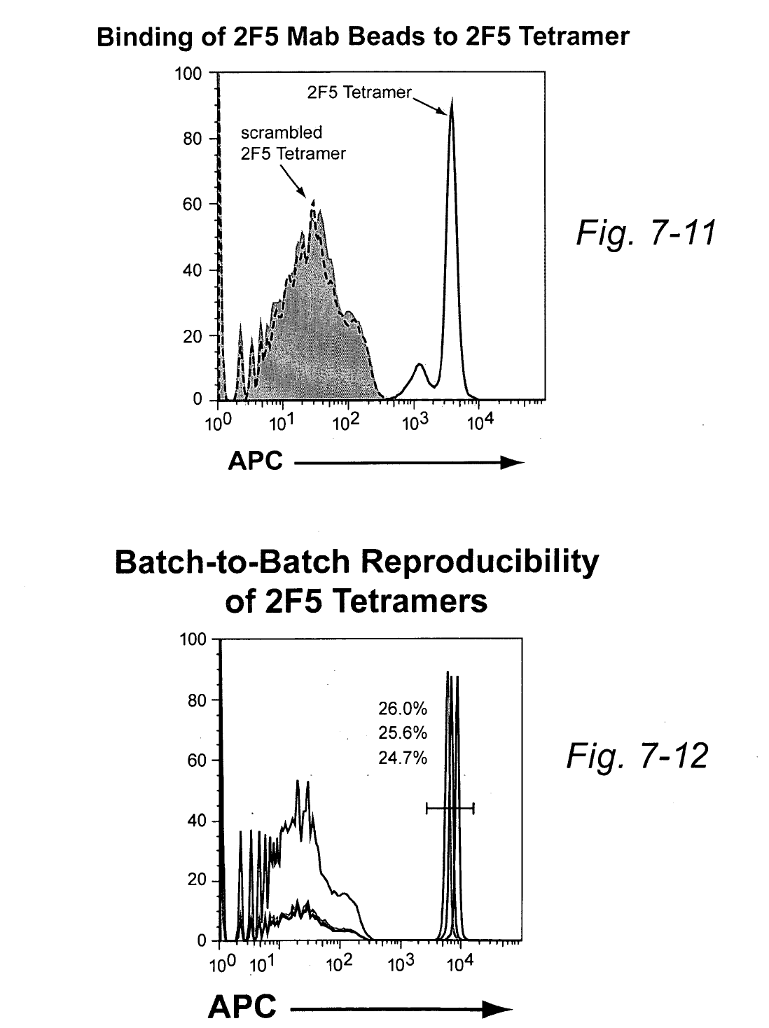

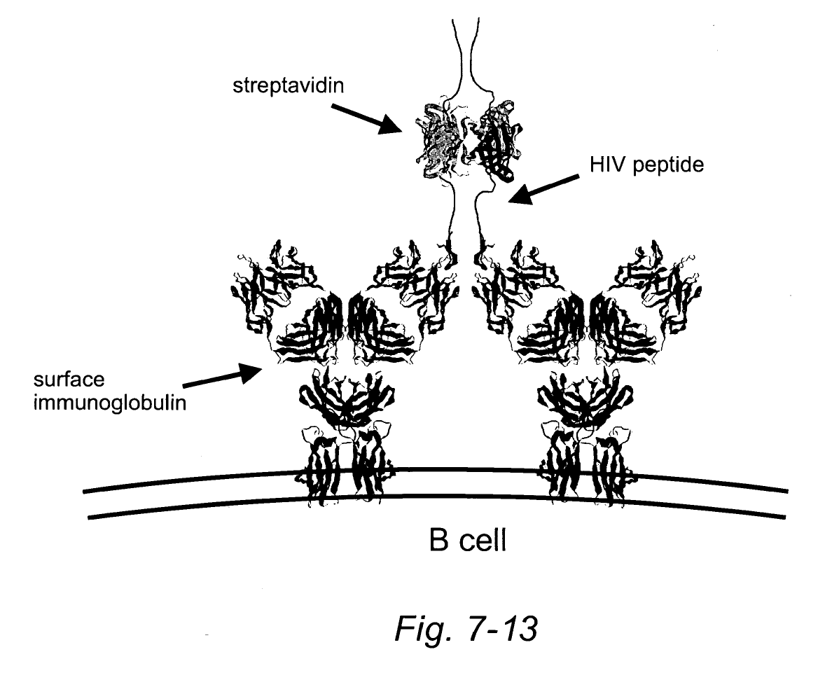

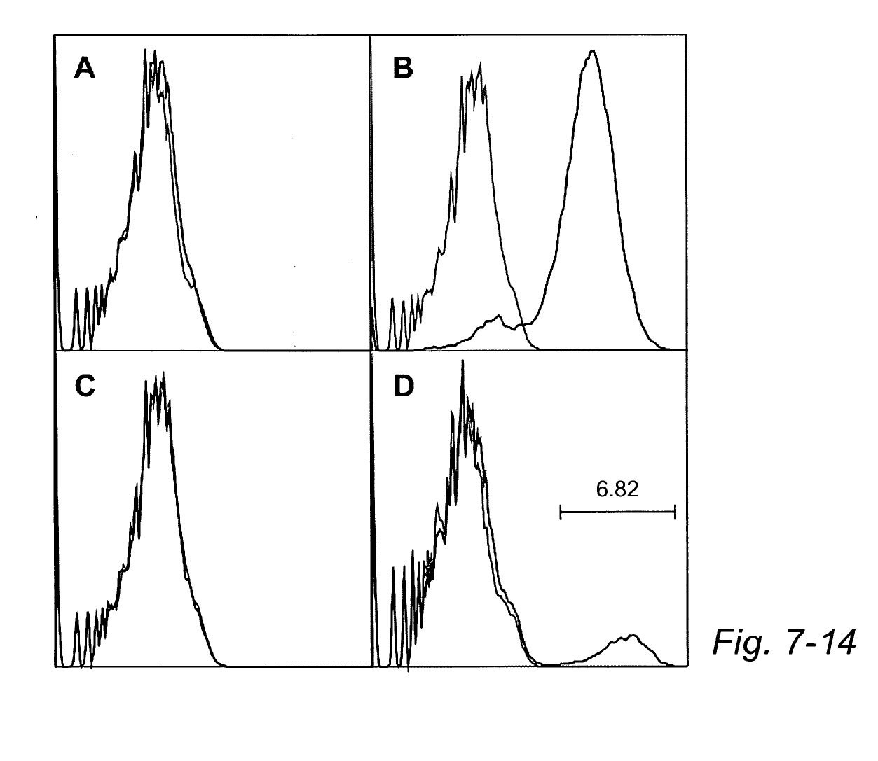

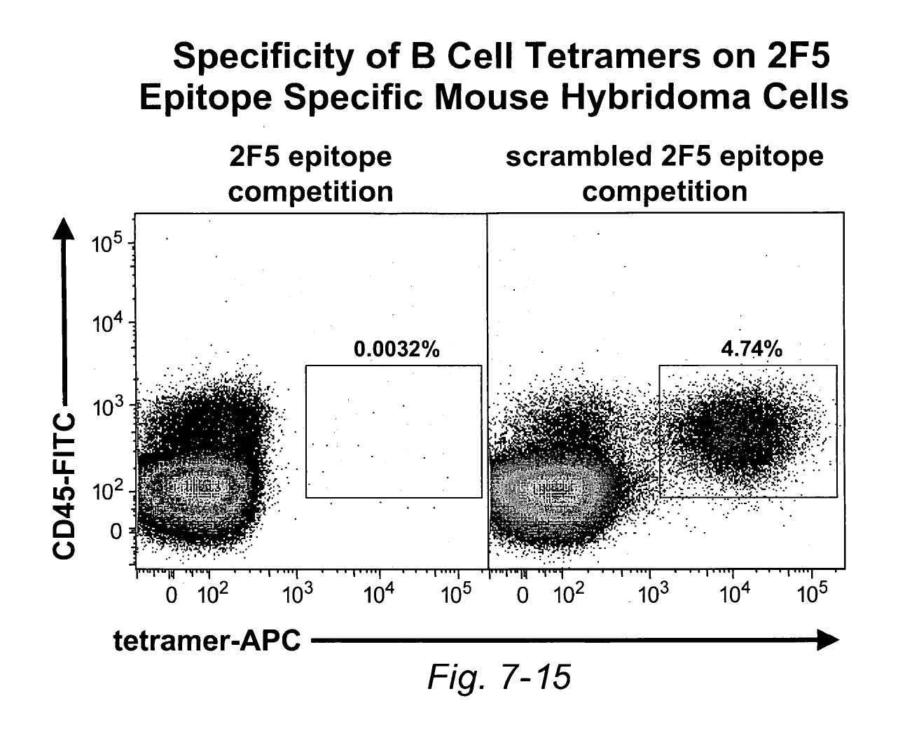

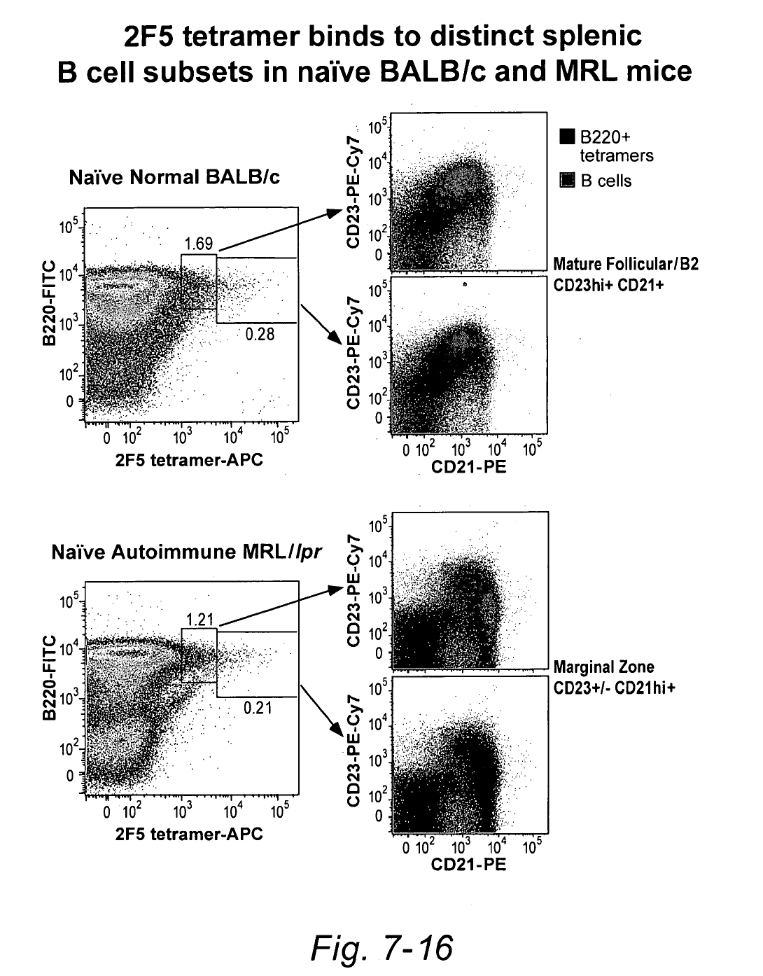

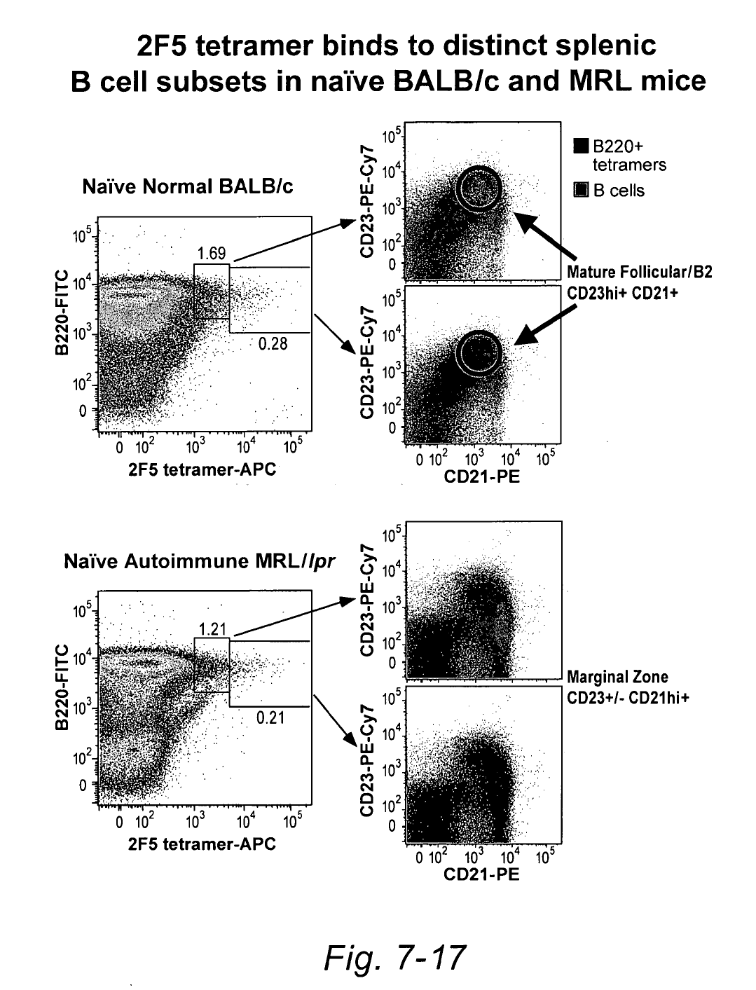

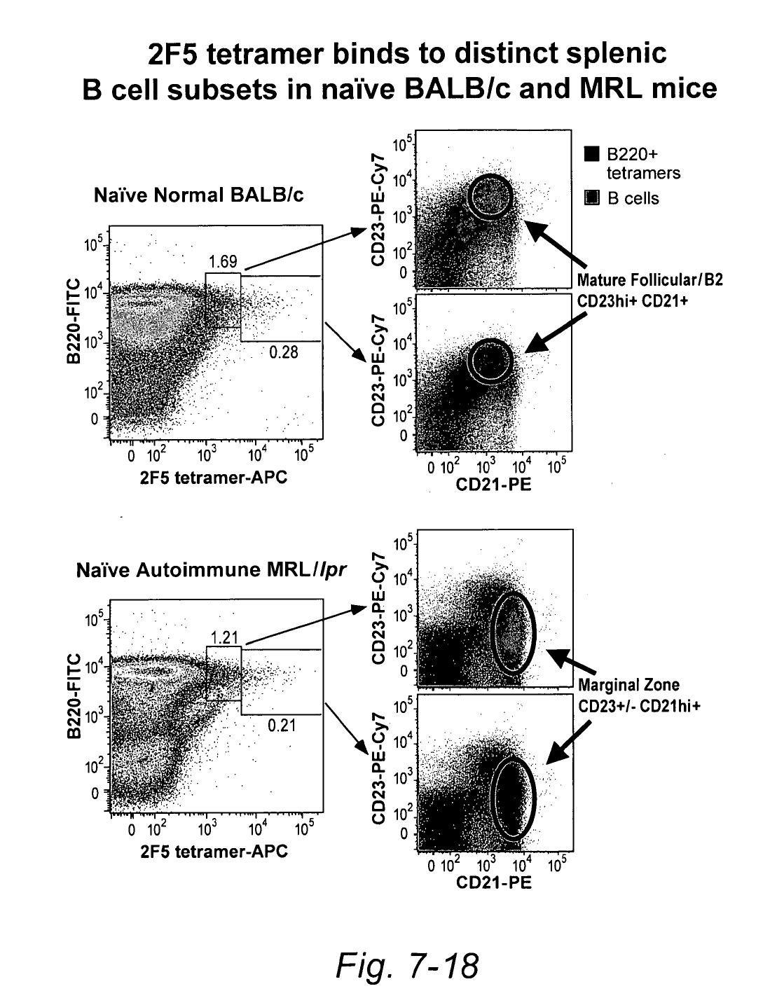

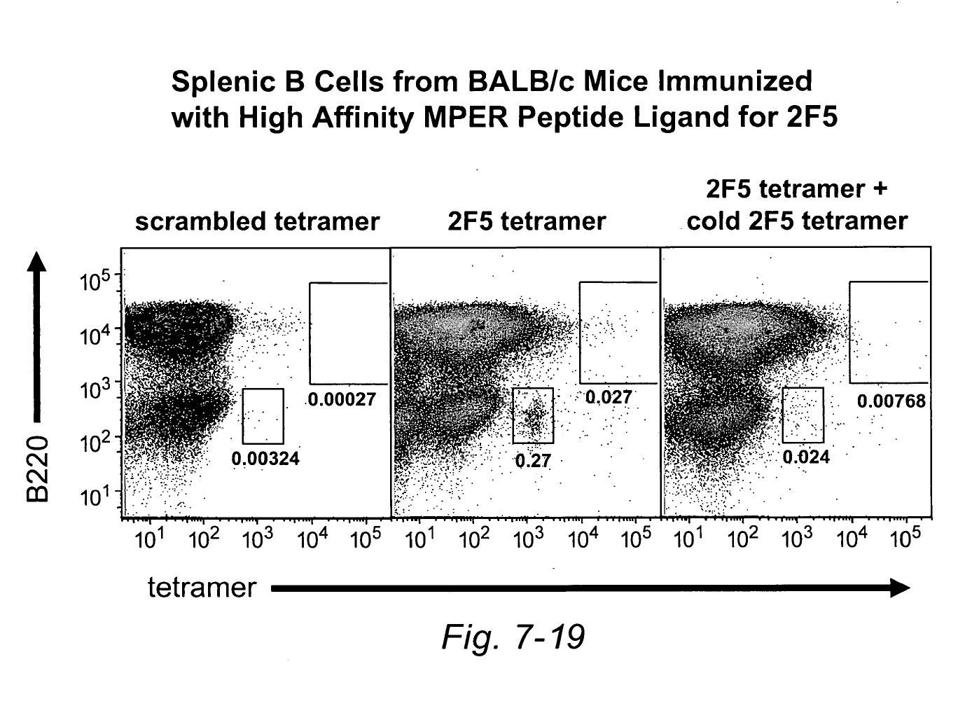

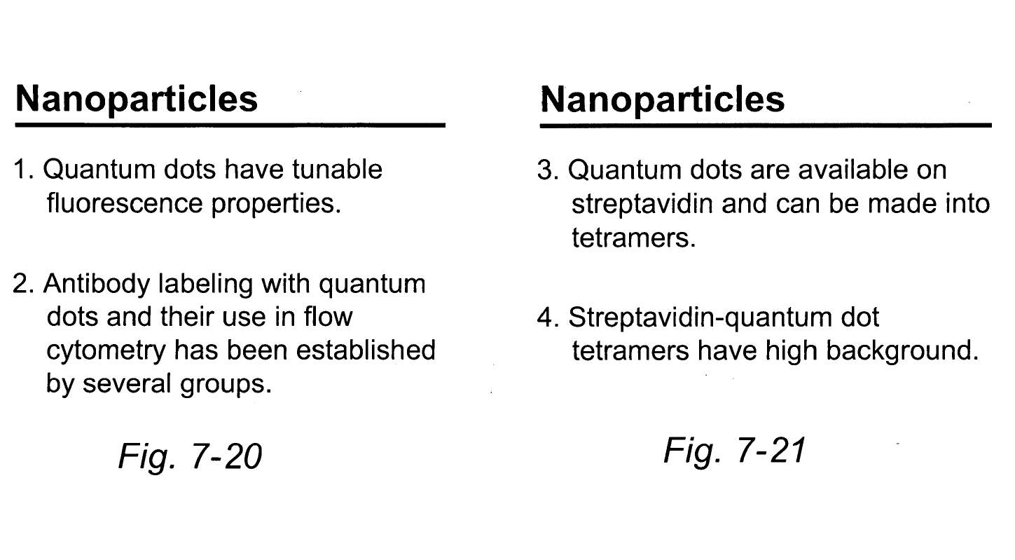

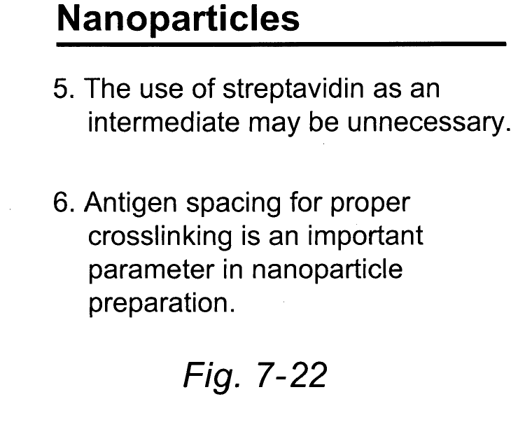

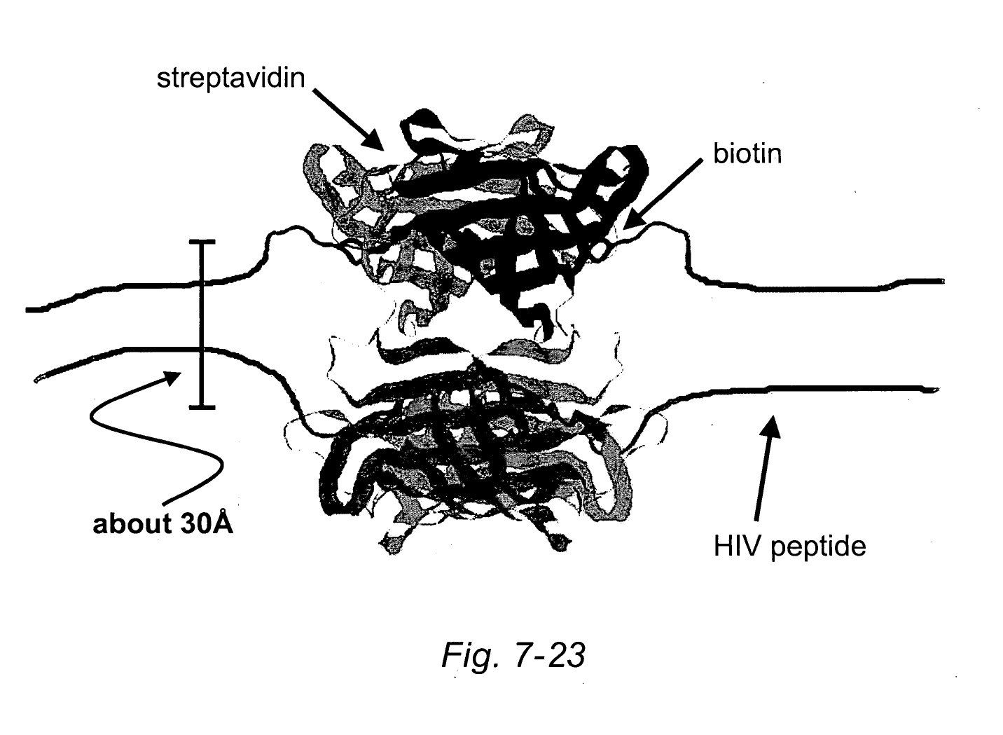

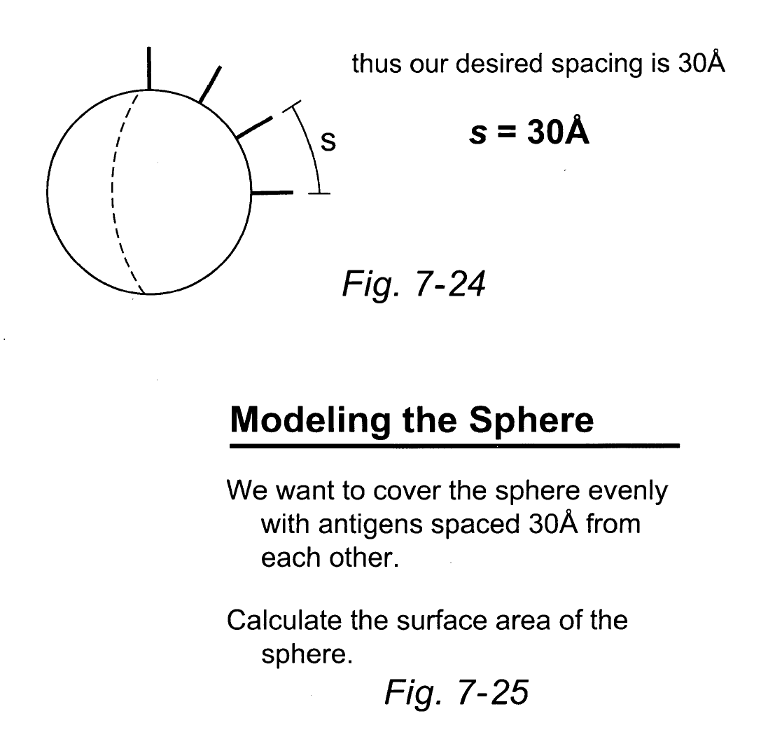

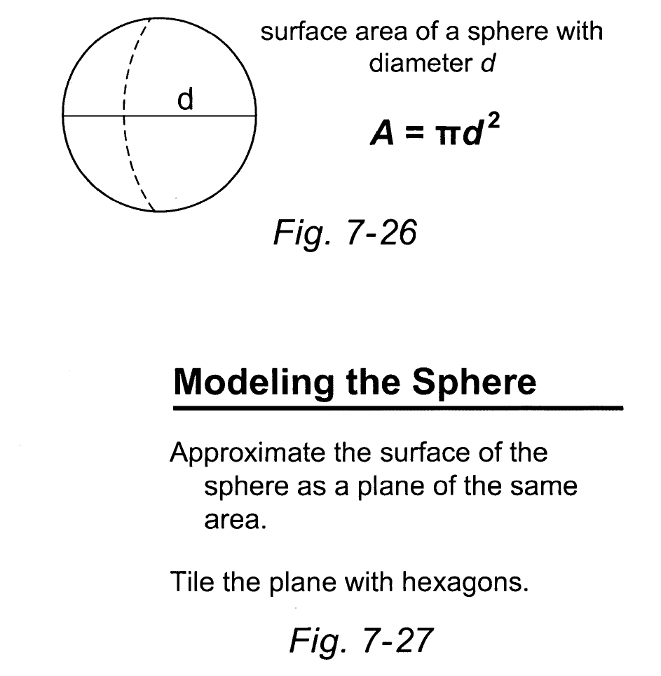

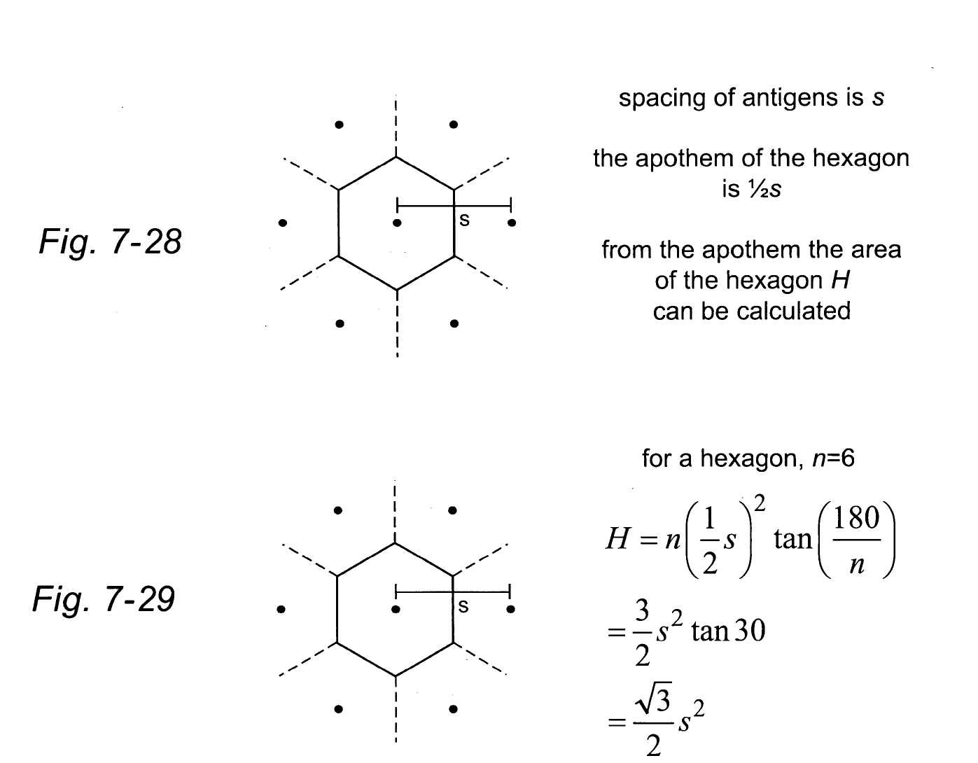

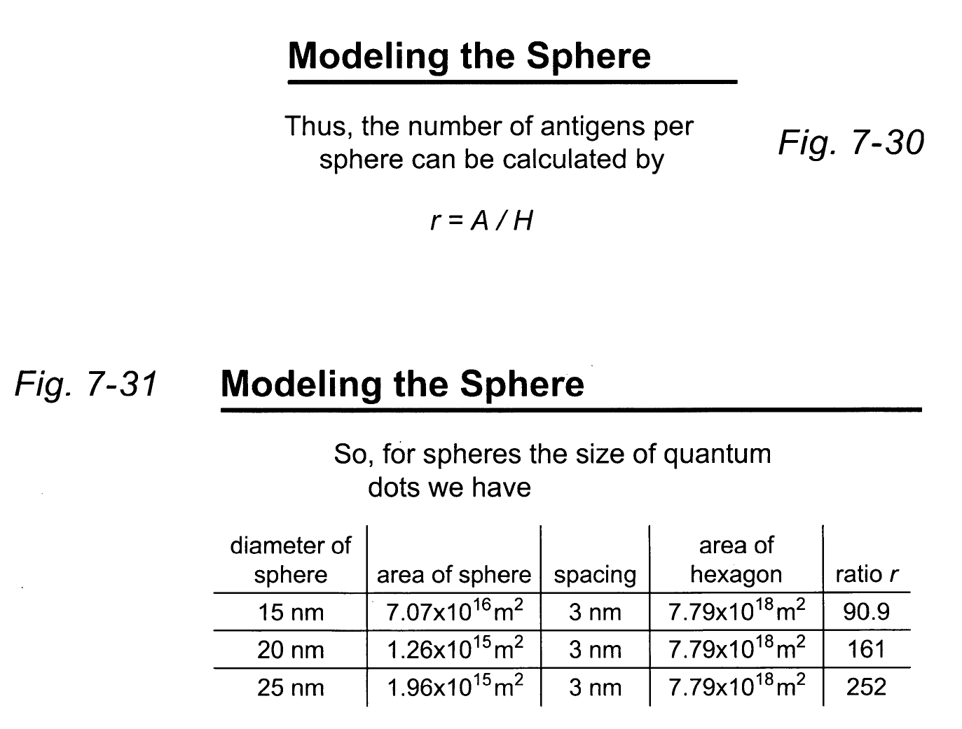

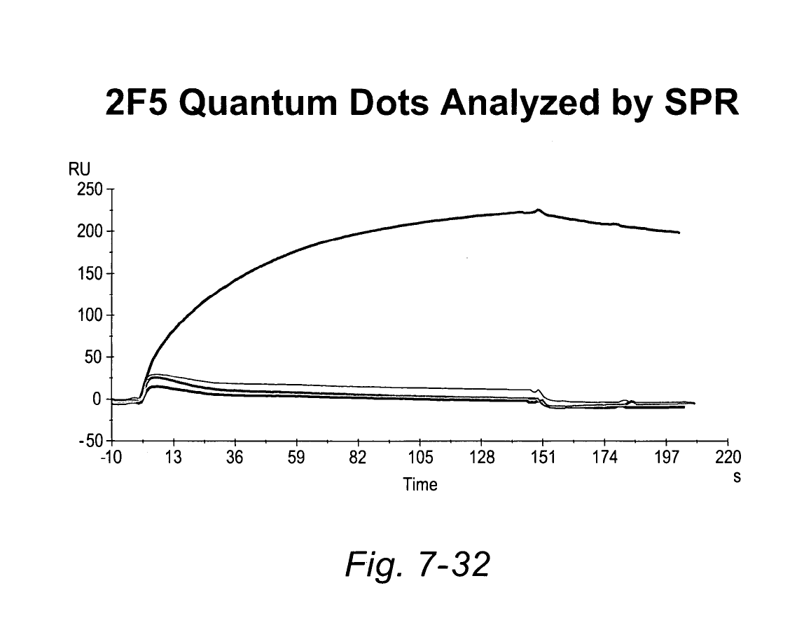

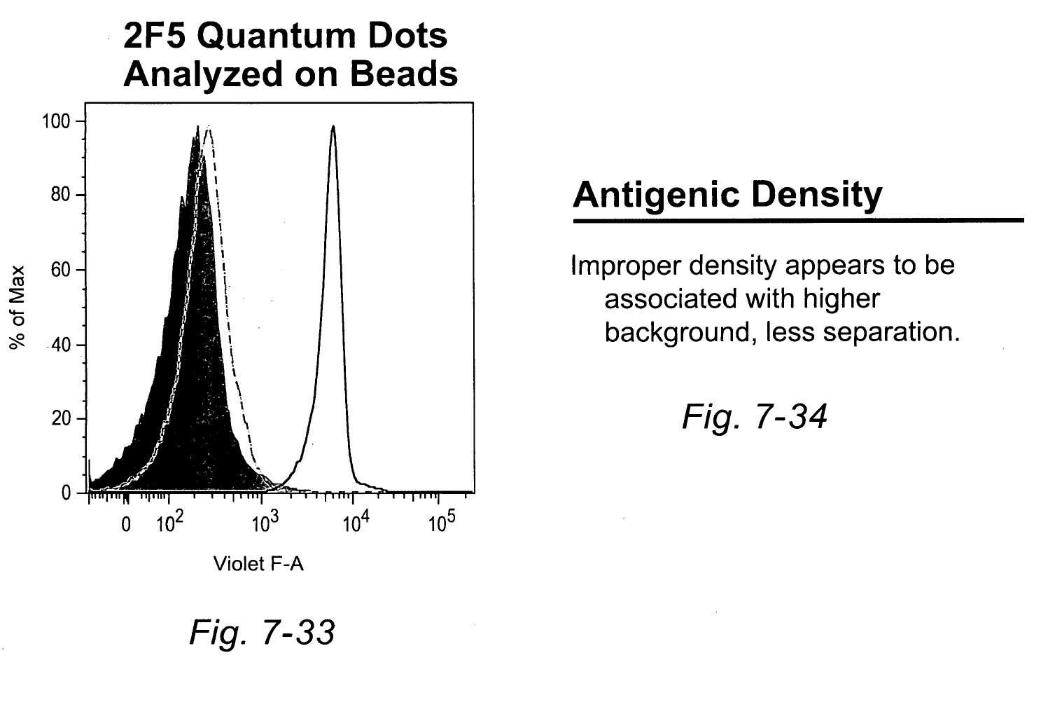

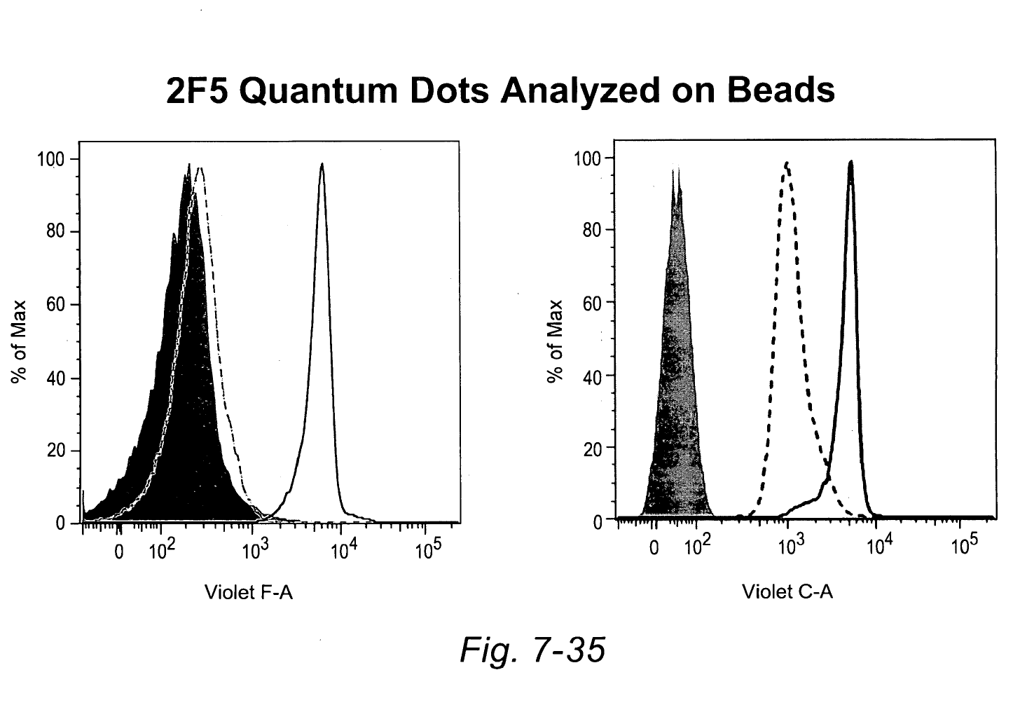

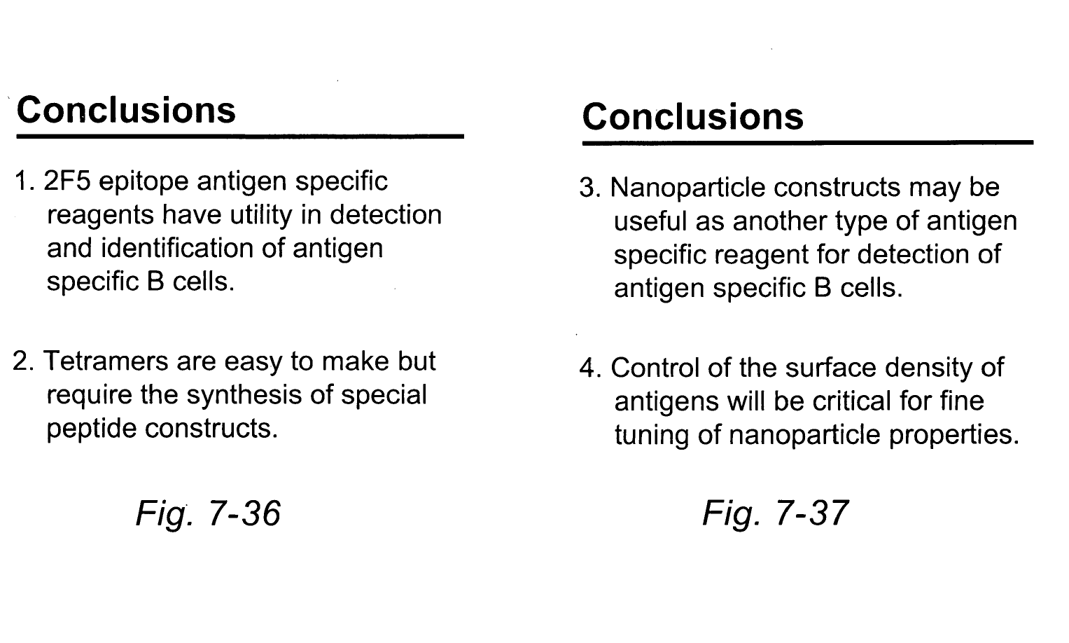

The nanoparticles were tested in a flow cytometry system using antibody coated beads that have been used to validate streptavidin-based detection reagents. FIG. 6 is a histogram plot of beads labeled with nanoparticles and analyzed using a Becton Dickinson LSR II flow cytometer equipped with a violet laser (405 nm) with appropriate detection optics. The filled grey curve shows unstained beads, the dashed curve shows beads labeled with a control antibody and stained with 2F5-epitope nanoparticles, and the black curve shows 2F5-epitope nanoparticles labeling beads coated with 2F5 antibody.

Summarizing, the invention allows for the creation of antigen-specific reagents that do not rely on the use of biotinylated reagents and streptavidin-fluorochrome conjugates. In addition, the approach allows for the creation of reagents inaccessible using traditional strategies. Some peptides and other molecules are insoluble or sparingly soluble in aqueous media. Those molecules can be solublized in organic solvents and reacted with nanoparticles that are resistant to those solvents. Thus molecules that cannot be made into streptavidin-based tetramers, such as highly hydrophobic proteins or peptides, may be accessible by this method.

Although nanoparticles have been used to label antibodies and other molecule specific probes (e.g., streptavidin), their use in creating antigen-specific reagents has not been described. This technology has the advantage of greater versatility over previous methods. These nanoparticles can be used to detect components of the immune system that recognize molecules of interest such as HIV-1 antigens and can be used to quantify, isolate, and study the immune system to solve the problem of immunity to HIV-1.

All documents and other information sources cited above are hereby incorporated in their entirety by reference.

What is claimed is:

1. A particle comprising a nanoparticle and peptide, polypeptide, protein, carbohydrate, lipid, or nucleic acid molecules, wherein said peptide polypeptide, protein, carbohydrate, lipid or nucleic acid molecules are attached to, and evenly spaced on, the surface of said nanoparticle.

2. The particle according to claim 1 wherein said nanoparticle is a semiconductor crystal.

3. The particle according to claim 2 wherein said semiconductor crystal comprises cadmium selenide or indium arsenide crystals.

4. The particle according to claim 1 wherein said nanoparticle comprises a biodegradable polymer, persistent non-fluorescent material or ferromagnetic metal.

5. The particle according to claim 1 wherein said peptide, polypeptide, protein, carbohydrate, lipid or nucleic acid molecules are covalently attached to said surface of said nanoparticle.

6. The particle according to claim 1 wherein said nanoparticle has a diameter of 5-50 nm.

7. The particle according to claim 6 wherein said nanoparticle has a diameter of 15-25 nm.

8. The particle according to claim 1 wherein antigens are attached to said surface of said nanoparticle.

9. The particle according to claim 8 wherein said spacing of said antigens on said surface of said nanoparticle is about 5-50 Å.

10. The particle according to claim 9 wherein said spacing of said antigens on said surface of said nanoparticle is about 30 Å.

11. The particle according to claim 8 wherein said antigens are HIV antigens.

12. A method of preparing an antigen-specific nanoparticle comprising affixing antigens to the surface of a nanoparticle under conditions such that said antigens are evenly spaced on the surface of said nanoparticle.

13. The method according to claim 12 wherein said method comprising contacting said antigens with said nanoparticles under conditions such that said antigens and said nanoparticles are coupled in a regiospecific manner.

14. The method according to claim 13 wherein the number of antigens per nanoparticle is r,
wherein:
$$r = \frac{A}{H} , \\ \begin{matrix}H = {n ( \frac{1}{2} s )}^{2} tan ( \frac{180}{n} ) \\ = \frac{3}{2} {s}^{2} tan 30 \\ = \frac{\sqrt{3}}{2} {s}^{2} ,\end{matrix}$$
$$and$$
A=πd²,
wherein d is the diameter of the nanoparticle, s is the spacing of the antigens and n=6.

15. A method of isolating antigen-specific B cells from a population of B cells comprising contacting said population with said particles according to claim 8 under conditions such that antigen-specific B cells present in said population bind to said particles to form antigen-B cell complexes and separating said complexes from said population of B cells.

16. The method according to claim 15 wherein said antigens are HIV antigens.

17. The method according to claim 15 wherein said population of B cells is derived from a patient infected with HIV.

18. An immunogen comprising said particles according to claim 8 and a carrier.

19. The immunogen according to claim 18 wherein said antigens are HIV antigens.

20. A method inducing an immune response in a patient comprising administering to said patient an amount of said particles according to claim 8 sufficient to induce said response.

21. The method according to claim 20 wherein said antigens are HIV antigens.

22. The method according to claim 21 wherein said patient is a human.
